(19) United States
(12) Patent Application Publication
(10) Pub. No.: US 2002/0045818 A1
(43) Pub. Date: Apr. 18, 2002

(54) Intergrated circuit for generating a high-voltage pulse for use in an ultrasound diagnostic system

(75) Inventor: Ki Jeon, Seoul (KR)

Correspondence Address: WIGGIN & DANA LLP, ATTENTION: PATENT DOCKETING, ONE CENTURY TOWER, P.O. BOX 1832, NEW HAVEN, CT, 06508-1832, US

(73) Assignee: Medison Co., Ltd

(21) Appl. No.: 09/909,521

(22) Filed: Jul. 20, 2001

(30) Foreign Application Priority Data
Oct. 12, 2000 (KR) 2000-59925

(51) Int. Cl. A61B 8/00

(52) U.S. Cl. 600/437

(57) ABSTRACT

An integrated circuit generates a high-voltage coded excitation pulse for use in producing a sharply defined diagnostic image in an ultrasound diagnostic system. The integrated circuit comprises at least two signal generator for generating a driving signal and a pulser, responsive to the driving signal, for creating a pulse sequence constructed by a combination of a first and a second voltage level, wherein the pulse sequence is made of a random code sequence.

### BACKGROUND OF THE INVENTION

1. Field of the Invention

The present invention relates to an ultrasound diagnostic system; and, more particularly, to an integrated circuit for use in the system for generating a high-voltage coded excitation pulse to be used in producing a sharply defined diagnostic image.

2. Description of the Related Art

An ultrasound diagnostic system is gaining popularity in medical applications for obtaining a diagnostic image of an anatomy in a human body. An ultrasound signal is generated by a transducer or an array of transducers, which convert electrical energy into acoustic energy for transmission into an object to be examined. The transducer may also receive echoes indicative of discontinuities or impedance variations on the ultrasound signal reflected from the object. The received echoes are processed by the transducer, i.e., converted to an electrical signal. The electrical signal is amplified and decoded to produce an image of the object.

Typically, in order to generate an ultrasound signal, a high-voltage pulse is advantageously used. Pulses would have a predefined voltage level, e.g., ranging from −80 V (volts) to +80 V, or from zero to +200 V. One should be able to control the voltage levels without causing unexpected damage or harm to a target object, specifically those within a human body. Details of the voltage level adjustment will be explained with reference to FIGS. 1 and 2 below.

FIG. 1 shows a block diagram of a unit 100 for transmitting and receiving ultrasound signals. The unit 100 comprises a transducer 110, a cable 120, a pulser 130, a limiter 140 and a pre-amplifier 150. The pulser 130 receives UP and DOWN signals from a signal generator (not shown). In response to the UP and DOWN signals, the pulser 130 generates high-voltage pulses for use in the generation of the ultrasound signals. The high-voltage pulses are outputted through the cable 120 to the transducer 110, which in turn. In response to the high-voltage pulses from the pulser 130, the transducer 110 generates and transmits ultrasound signals to an object (not shown) within a human body to be examined. The ultrasound signals generated by the transducer 110 would have a resonant frequency and acoustic energy as determined by electrical energy carried by the high-voltage pulses.

Transmitted ultrasound signals are reflected and received by the transducer 110. The transducer 110 converts the reflected ultrasound signals back to electrical signals corresponding thereto. The electrical signals are provided via the cable 120 to the limiter 140 before outputted to the pre-amplifier 150. The pre-amplifier 150 amplifies the electrical signals to a predetermined level to transmit it to a signal processing unit (not shown).

The pulser 130 is required to provide a current of 2 A (ampere) for generating the high-voltage pulses, since it is connected to the cable 120 of, e.g., 400 pF (pico-Faraday) having a capacitive load of the transducer 110 and to the limiter 140 having an input impedance of, e.g., 100 Ω (ohm). It is also necessary that the rising and falling times of the high-voltage pulses should be less than 20 ns (nanosecond), because the pulse frequency suitable for generating the ultrasound signals must be at least 12 MHz (Mega-Hertz). In other words, the high-voltage pulse width should be within 40 ns. Conventional pulsers commonly include a step-up transformer in order to satisfy the aforementioned requirements.

FIG. 2 illustrates a circuit diagram of the pulser 130 according to the prior art. It is constructed by including a step-up transformer 132. For the convenience of explanation, the left portion, i.e., a primary winding side of the step-up transformer 132 is referred to as an input part, whereas the right portion, i.e., a secondary winding side thereof is called an output part. And, for the purpose of simplicity, the principle of an operation of the transformer 132 is omitted as it is well known in the art.

UP and DOWN signals are provided to the pulser 130 through different paths, wherein the UP and DOWN signals are each a digital signal having a voltage level ranging 0 to 5 V, for instance. The UP signal is provided to the base of a transistor 134 through a capacitor C1, while the DOWN signal is inputted to the base of a transistor 136 via a capacitor C2. The collectors of the transistors 134 and 136 are connected to the primary winding of the step-up transformer 132. As the UP and DOWN signals are inputted, the step-up transformer 132 serves as a level shifter to allow CMOS (Complementary Metal Oxide Semiconductor) transistors 138 and 140 to generate a high-voltage pulse, the transistors 138 and 140 being connected to the secondary winding of the transformer 132. Such generated high-voltage pulse is then transmitted to the cable 120 shown in FIG. 1.

Specifically, the pulser 130 according to the prior art generates a single high-voltage or a burst high-voltage pulse shown in FIG. 6, depending on a part of an object within the human body to be examined or a color diagnostic image thereof. In the single high-voltage pulse or the burst high-voltage pulse, the width of each of logic high and logic low thereof has a range from maximum 500 ns to minimum 30 ns. In this circuit arrangement, when the UP signal changes from logic low to logic high, the output voltage from the pulser 130 would reach a voltage Vpp applied to the CMOS transistor 138. Although the UP signal remains in logic high for a certain time, the output voltage Vpp is no longer maintained after that time and thus returns to 0 V. Upon transiting from logic low to logic high of the UP signal, a voltage across a resistor R4 returns from Vpp to zero. The resistor R4 is connected to Vpp, to the secondary winding of the step-up transformer 132 and to the gate of the CMOS transistor 138. When the DOWN signal is changed from logic low to logic high, a similar phenomenon as described above is occurred. As well known in the art, these results from an electromotive force of the transformer 132, wherein the electromotive force is created only by a current alternation flowing on the primary winding of transformer 132.

Recently, in order to obtain a sharply defined diagnostic image, a method for combining transducers or filtering reflected ultrasound signals has been studied in depth. As well as such method, a circuit device for generating high-voltage pulses for use in generating ultrasound signals has been also studied in further detail. It is well known to those skilled in the art that high-voltage coded excitation pulses containing an arbitrary code sequence are very effective in producing the sharply defined a random pulse sequence which is comprised of first and second voltage levels.

In accordance with another aspect of the present invention, there is provided an integrated circuit for providing an electrical signal for use in generating an ultrasound signal in an ultrasound diagnostic system, comprising: at least two signal generators for generating a control signal; and a pulser, responsive to the control signal, for providing the electrical signal having first and second signal levels, wherein the electrical signal includes a random code represented by a combination of the first and second signal levels.

### BRIEF DESCRIPTIONS OF THE DRAWINGS

The above and other objects and features of the present invention will become apparent from the following description of a preferred embodiment given in conjunction with the accompanying drawings, in which:

FIG. 1 shows a block diagram of a typical unit for transmitting and receiving an ultrasound signal;

FIG. 2 depicts a circuit diagram of a pulser according to the prior art;

FIG. 3A exemplifies a circuit diagram of a pulser in accordance with the present invention;

FIGS. 3B and 3C illustrate diagrams for explaining the operation of the pulser shown in FIG. 3A;

FIG. 4 presents a waveform of a single pulse generated by the pulser in accordance with the present invention;

FIG. 5A is a diagram for comparing the waveform of a single pulse generated by the pulser shown in FIG. 2 with that of the single pulse generated by the pulser shown in FIG. 3A;

FIG. 5B is a diagram of a high-voltage coded excitation pulse generated by the pulser in accordance with the present invention; and

FIG. 6 represents a diagram for explaining single, burst, and high-voltage coded excitation pulses.

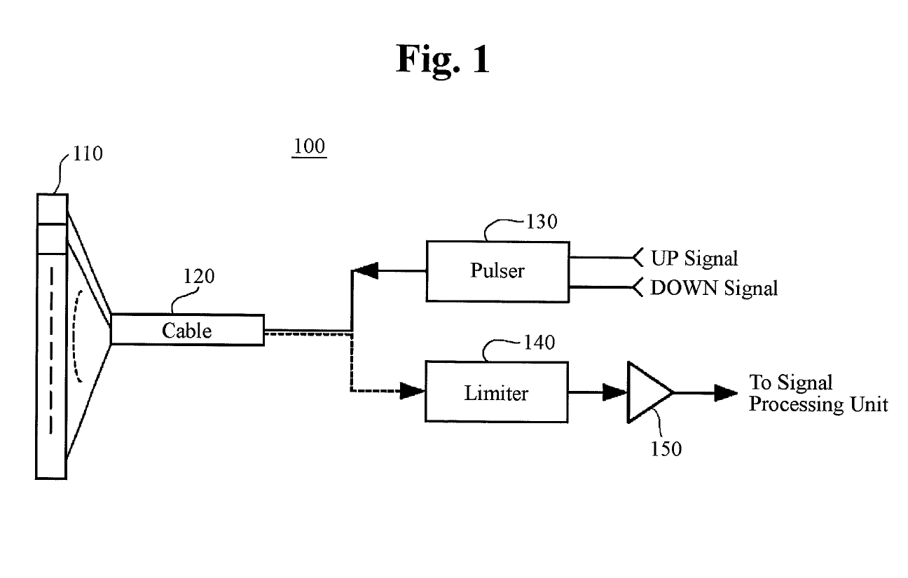

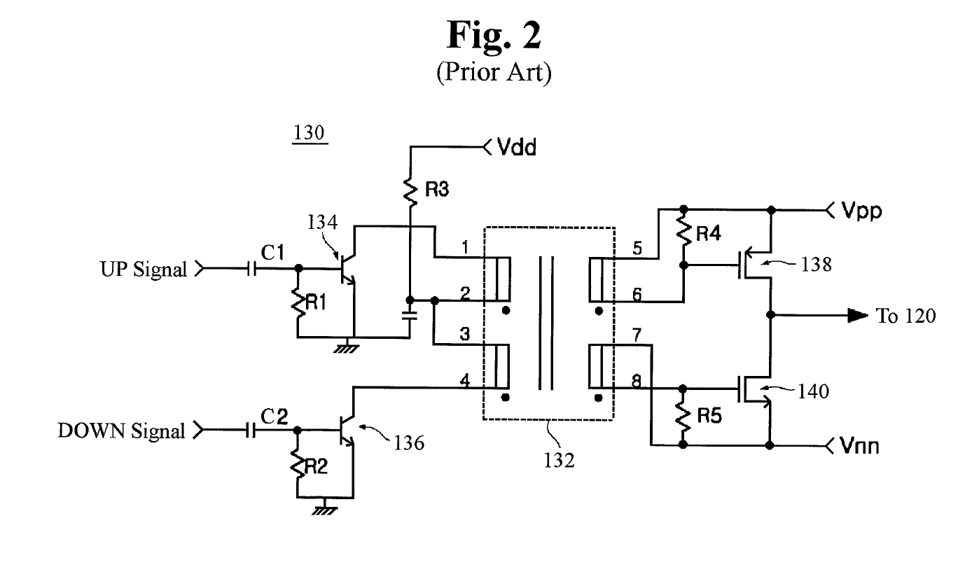

### DETAILED DESCRIPTION OF THE PRESENT INVENTION

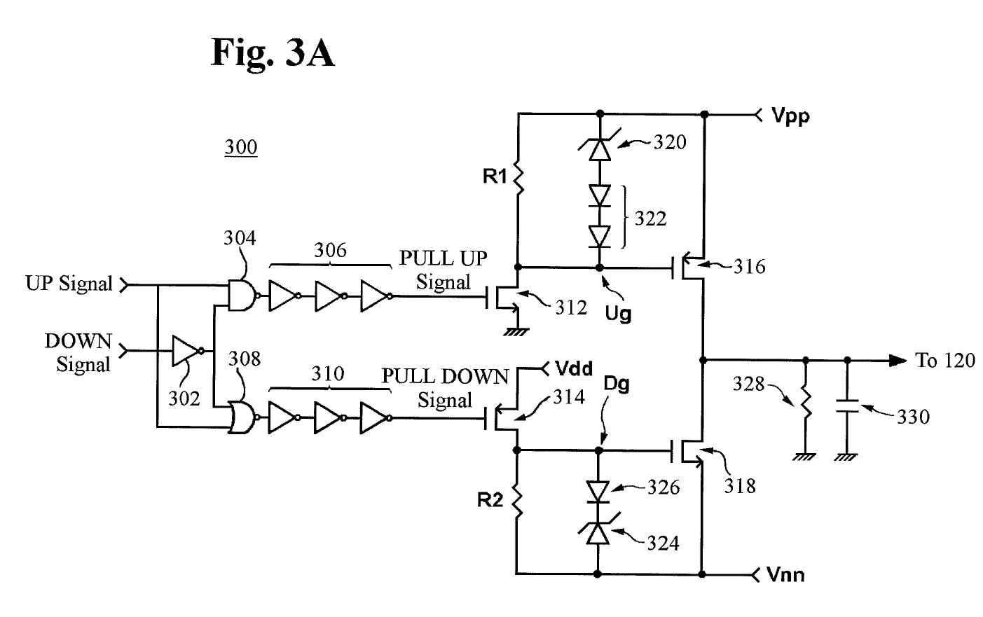

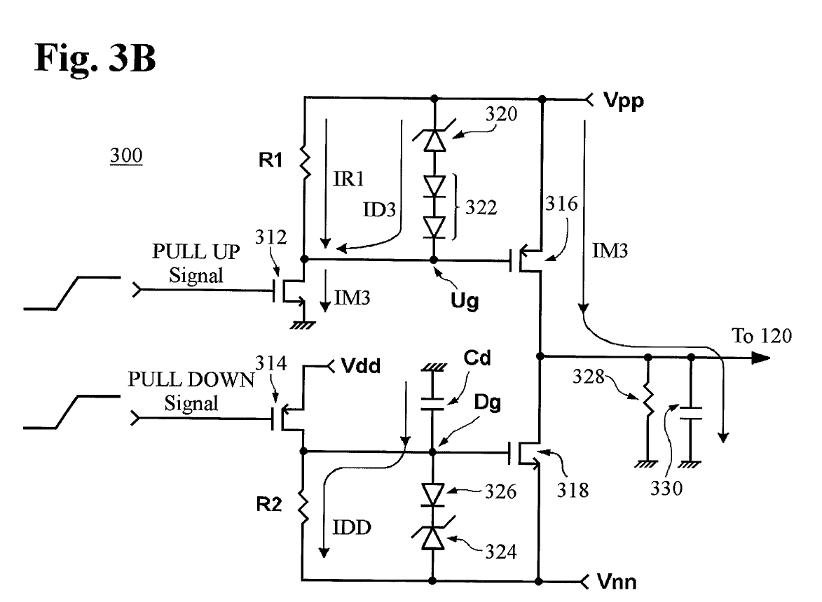

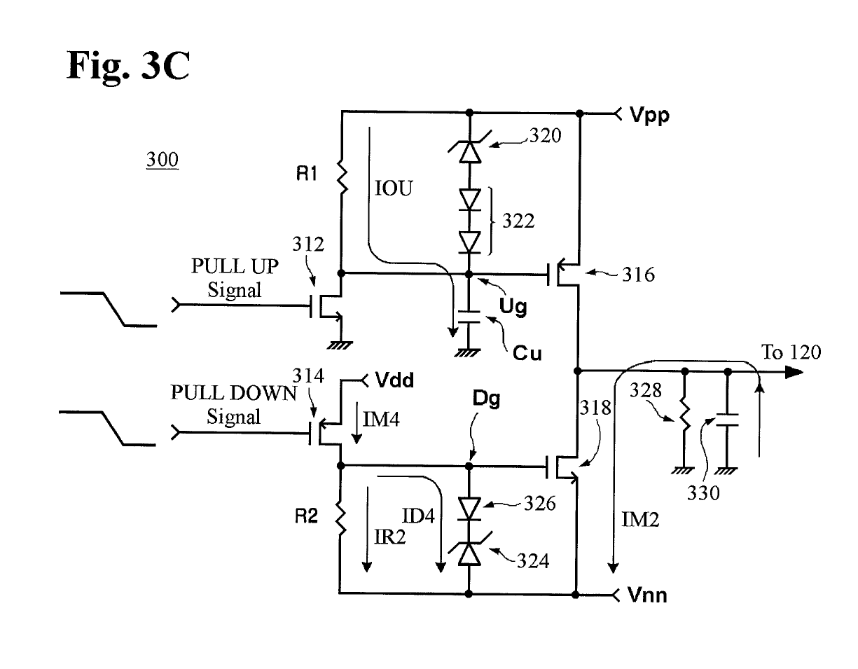

FIG. 3A shows a circuit diagram of a pulser 300 in accordance with the present invention. An UP signal is inputted to an input port of a NAND gate 304 as well as an input port of a NOR gate 308, while a DOWN signal is provided to an inverter 302. The output of the inverter 302 is commonly coupled to the other input ports of the NAND gate 304 and the NOR gate 308. Like the prior art, each of the UP and DOWN signals is a digital signal having a voltage level of 0 to 5 V, for instance. The output signal of the pulser 300 becomes logic high when the UP signal is logic high, and changes to logic low when the DOWN signal is logic low.

The output signal of the NAND gate 304 is transmitted through a series of inverters 306, to finally become a PULL UP signal. Similarly, the output signal of the NOR gate 308 is provided to a series of inverters 310 to finally become a PULL DOWN signal. The PULL UP and PULL DOWN signals are provided to the gate of a high-voltage n-type CMOS transistor 312 and to the gate of a high-voltage p-type CMOS transistor 314, respectively. These have a voltage level of, e.g., 0 to 5 V and are used to drive the high-voltage CMOS transistors 312 and 314, respectively.

The source of the high-voltage n-type CMOS transistor 312 is grounded and its drain is coupled to one terminal of a resistor R1 of, e.g., 60 Ω, to the cathode of a series of diodes 322 and to the gate of a high-voltage CMOS transistor 316. The other terminal of the resistor R1 is connected to the cathode of a Zener diode 320 and to Vpp; and the anode of the diodes 322 is coupled to that of the Zener diode 320. The cathode of the Zener diode 320 is connected to Vpp. Vpp has a voltage level of +80 V, for instance. The source of the high-voltage p-type CMOS transistor 314 is connected to Vdd having a voltage level of +5 V, for instance. The drain thereof is coupled to one terminal of a resistor R2, to the anode of a diode 326 and to the gate of a high-voltage CMOS transistor 318. The cathode of the diode 326 is coupled to that of a Zener diode 324. The anode of the Zener diode 324 and the other terminal of the resistor R2 are connected to Vnn having a voltage level of, −80 V, for instance. It should be interpreted and understood that the term “high-voltage” used here means an absolute voltage value greater than a range of 0 to +5 V, but is not limited to the exemplary range of, +80 V to −80 V.

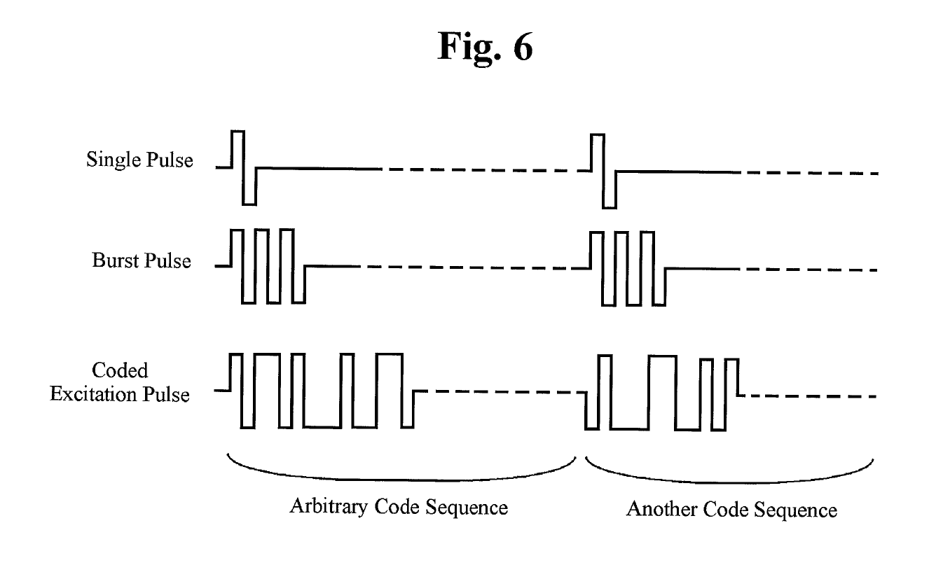

Now, the details of the pulser 300 in accordance with the present invention will be described with reference to FIGS. 3B to 6.

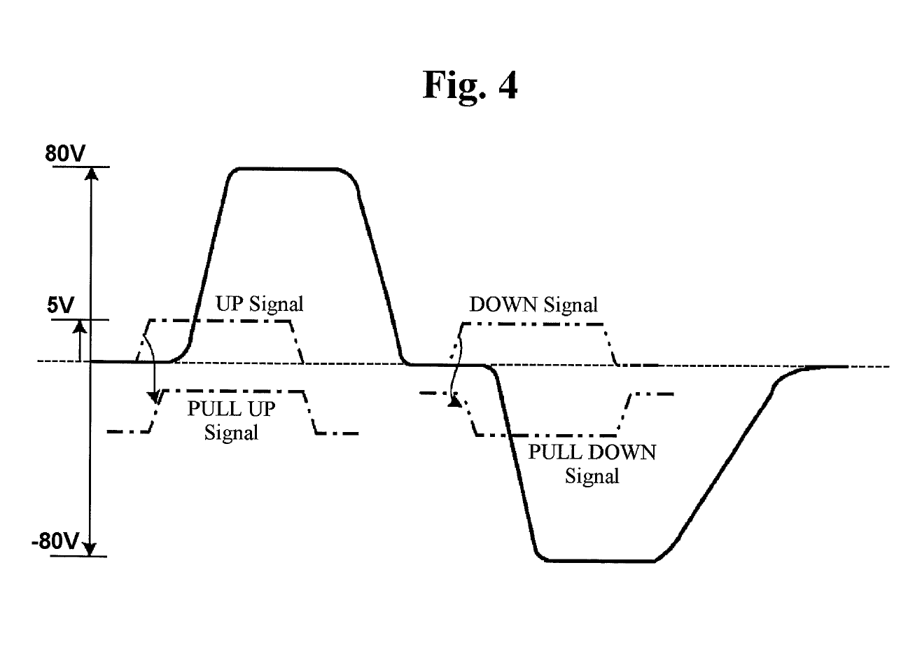

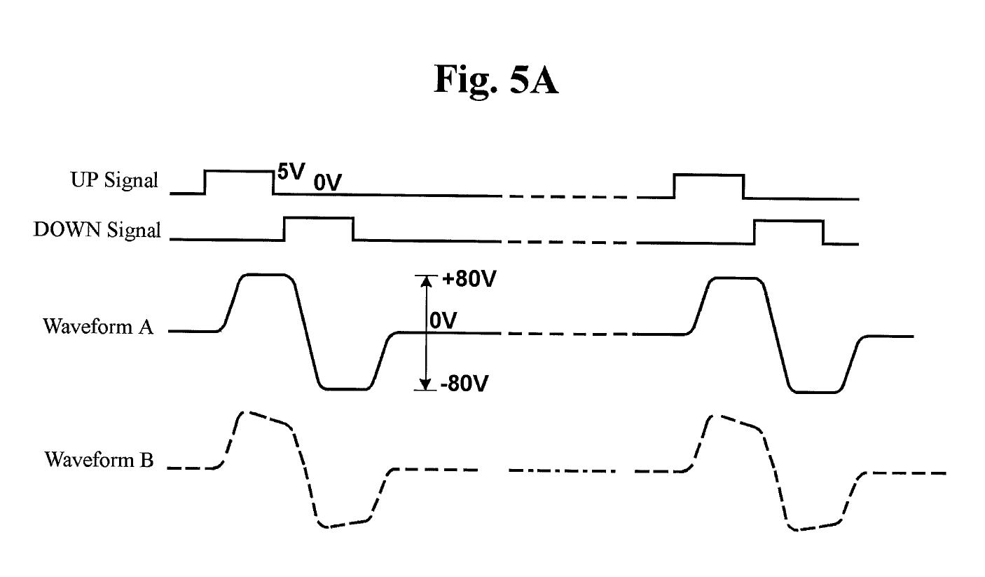

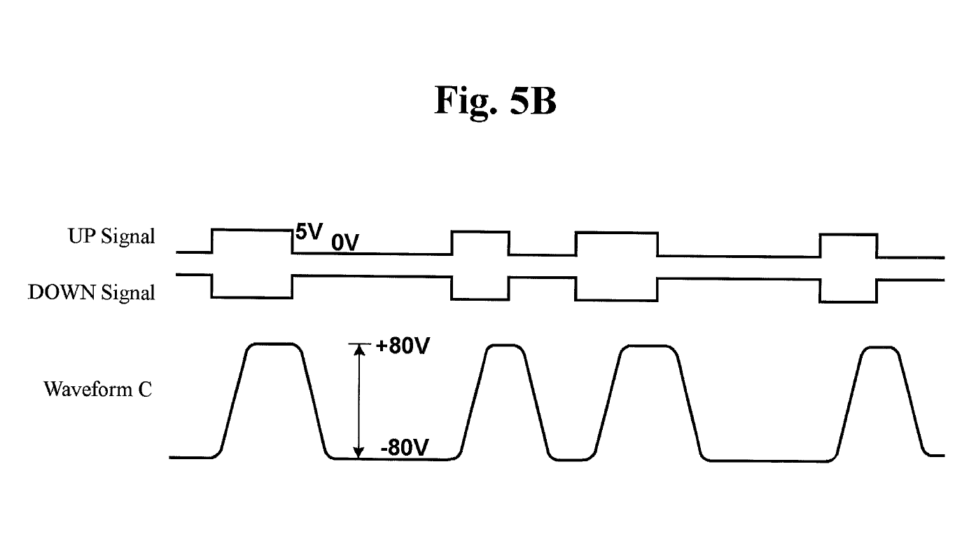

In FIG. 3B, when the UP signal is logic high and the DOWN signal is logic low, the PULL UP and PULL DOWN signals change from logic low to logic high. In response to the PULL UP signal, the CMOS transistor 312 is turned on so that a current IR1 flows through the resistor R1, while a current ID3 flows through the Zener diode 320 and the diodes 322. Then, a voltage about 8 V (the sum of the breakdown voltage of the Zener diode 320 and the turn-on voltages of the diodes 322) is seen between Vpp and a node Ug, which is at the gate of a transistor 316. That voltage is sufficient to drive the CMOS transistor 316. On the other hand, the CMOS transistor 314, responsive to the PULL DOWN signal of logic high, is turned off to discharge a parasitic capacitor Cd on a node Dg such that a current IOD flows toward Vnn through the resistor R2. At this time, a voltage level at the node Dg, which is at the gate of a transistor 318 becomes Vnn. Thus, the CMOS transistor 318 is turned off. As a result, a current IM3 flows from Vpp through the CMOS transistor 316 to a load capacitor 330. The load capacitor 330 is charged by the current IM3, so that the output voltage of the pulser 300 rises to Vpp of +80 V, as shown in FIG. 4 and a waveform A of FIG. 5A. It should be noted that the Zener diode 320 and the diodes 322 serve to prevent the gate of the CMOS transistor 316 from breaking down due to an issuance of an undesirable high-voltage.

When the UP signal is logic low and the DOWN signal is logic high, the PULL UP and PULL DOWN signals change from logic high to logic low as shown in FIG. 3C. In responds to the PULL UP signal of logic low, the CMOS transistor 312 is turned off so that a parasitic capacitor Cu on the node Ug is charged by a current IOU flowing through the resistor R1. At this time, the voltage at the node Ug becomes Vpp. Thus, there is no sufficient voltage difference to drive the CMOS transistor 316. As a result, the CMOS transistor 316 is turned off. On the other hand, the CMOS transistor 314, responsive to the PULL DOWN signal of logic low, is turned on so that a current IM4 flows from the source of the CMOS transistor 314 to the drain thereof. The current IM4 is divided into two, one of which, i.e., a current IR2 flows via the resistor R2 and the other of which, i.e., a current ID4 flows via the diode 326 and the Zener diode 324 to Vnn. As a result, the voltage at the node Dg rises to, approximately, 6.5 V from Vnn. Since this voltage of 6.5 V is also between the gate and the source of the CMOS transistor 318, the CMOS transistor 318 becomes conductive.

As described above, when the CMOS transistor 316 is turned off and the CMOS transistor 318 is turned on, a current IM2 flows toward Vnn through the CMOS transistor 318 as the load capacitor 330 begins to discharge. As a result, the output voltage of the pulser 300 falls to Vnn of −80 V, as shown in FIG. 4 and a waveform A of FIG. 5A. It should be noted that the Zener diode 324 and the diode 326 serve to prevent the gate of the CMOS transistor 318 from breaking down by an undesirable high-voltage.

When both of the UP and DOWN signals are logic low, the PULL UP signal would be logic low and the PULL DOWN signal would be logic high. Then, the voltage at the node Ug becomes Vpp, while the voltage at the node Dg becomes Vnn. Thus, both of the CMOS transistors 316 and 318 are turned off and a voltage across a load resistor 328 falls to 0 V, as shown in a waveform A of FIG. 5A. Consequently, the output voltage of the pulser 300 becomes 0 V.

In case that the PULL UP and PULL DOWN signals are either logic high or logic low for a predetermined time, the output voltage of the pulser 300 remains in its current logic state until the PULL UP and PULL DOWN signals change to the other logic level, as shown in a waveform C of FIG. 5B. Therefore, the pulser 300 in accordance with the present invention could generate a high-voltage coded spirit and the scope of the claims appended hereto.

What is claimed is:

1. A circuit for generating a pulse to be used in producing an ultrasound image, comprising:
at least two means for generating a driving signal; and
means, responsive to the driving signal, for creating a random sequence of pulses comprised of first and second voltage levels.

2. The circuit according to claim 1, wherein the random sequence of pulses is created based on the driving signal.

3. The circuit according to claim 1, wherein the driving signal has logic high and logic low states.

4. The circuit according to claim 1, wherein said at least two means for generating the driving signal and the means for creating the random sequence of pulses are integrated on one unit.

5. The circuit according to claim 1, wherein the first and second voltage levels have positive and negative voltage levels, respectively.

6. The circuit according to claim 4, wherein the means for creating the random sequence of pulses includes at least two switching means operating in response to the driving signal.

7. The circuit according to claim 4, wherein the means for generating the driving signal includes at least one logic gate and a plurality of inverters connected electrically to said at least one logic gate.

8. The circuit according to claim 7, wherein said at least one logic gate is one of a NAND gate and a NOR gate.

9. The circuit according to claim 6, wherein said at least two switching means are each a CMOS (Complementary Metal Oxide Semiconductor) transistor.

10. The circuit according to claim 6, wherein the means for creating the random sequence of pulses further includes more than one diode for preventing from breaking down said at least two switching means connected electrically thereto.

11. An integrated circuit for providing an electrical signal for use in generating an ultrasound signal in an ultrasound diagnostic system, comprising:
at least two means for generating a control signal; and
means, responsive to the control signal, for providing the electrical signal having first and second signal levels,
wherein the electrical signal includes a random code represented by a combination of the first and the second signal levels.

12. The integrated circuit according to claim 11, wherein the random code is used for producing a diagnostic image, wherein the diagnostic image represents an internal shape of an object to be examined.

13. The integrated circuit according to claim 12, wherein the random code is generated based on the control signal.

14. The integrated circuit according to claim 11, wherein the electrical signal has the form of a pulse sequence.

15. The integrated circuit according to claim 11, wherein the control signal has logic high and logic low states.

16. The integrated circuit according to claim 11, wherein the means for providing the electrical signal includes at least two switching means operating in response to the control signal.

17. The integrated circuit according to claim 11, wherein said at least two means for generating the control signal include at least one logic gate and a plurality of inverters connected electrically to said at least one logic gate.

18. The integrated circuit according to claim 17, wherein said at least one logic gate includes one of a NAND gate and a NOR gate.

19. The integrated circuit according to claim 16, wherein said at least two switching means are each a CMOS transistor.

20. The integrated circuit according to claim 16, wherein the means for providing the electrical signal further includes more than one diode for preventing from breaking down said at least two switching means connected electrically thereto.
